(19) United States
(12) Patent Application Publication
(10) Pub. No.: US 2010/0286501 A1
(43) Pub. Date: Nov. 11, 2010

(54) METHOD AND APPARATUS FOR MONITORING AN OBJECT OF INTEREST IN A MEDICAL SYSTEM

(75) Inventors: Dayu Chen, Shanghai (CN); Jonathan Paul Schultz, Shanghai (CN); Ming Yan, Shanghai (CN)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/678,315

(22) PCT Filed: Sep. 16, 2008
(86) PCT No.: PCT/IB2008/053735

§ 371 Date: Jul. 19, 2010

(30) Foreign Application Priority Data
Sep. 18, 2007 (CN) 200710153968.8

(51) Int. Cl. A61B 5/05; A61B 8/00

(52) U.S. Cl. 600/410; 600/437

(57) ABSTRACT

The invention relates to a method and apparatus for monitoring an object of interest (100). According to the invention, a cushion (104) is inflated such that the inflated cushion extends inwards to envelop the object of interest (100), whereupon the pressure exerted on the object of interest (1600) by the inflated cushion (104) is measured and a signal is output to stop inflating when the pressure reaches a predefined threshold. After that, the volume of the inflated cushion is measured and the size of the object of interest (100) is derived from the volume of the inflated cushion (104). In this way, the invention can keep the object of interest still during monitoring. Furthermore, the size of the object of interest can be used for imaging reconstruction to improve the quality of reconstructed images.

### FIELD OF THE INVENTION

The invention relates to the monitoring of an object of interest, particularly to a method and apparatus for monitoring an object of interest in a medical imaging system such as a magnetic induction tomography system.

### BACKGROUND OF THE INVENTION

Magnetic induction tomography (MIT) is a conductivity distribution measuring technology that can be used for many biomedical applications. For example, blood has a higher conductivity than normal brain tissue, and checking the change of conductivity distribution in the brain can help identify brain hemorrhage.

In a MIT system for imaging reconstruction, a typical measurement takes around 10 seconds or even longer, depending on the application. A movement during measurement may introduce errors into measurements and even ruin the measurement data. It is very important to keep the object of interest to be measured immobilized during monitoring. Furthermore, the size of the object of interest is an important factor that influences the quality of the reconstructed conductivity distribution image. Particularly the size of the object of interest, such as the human head, is different from one case to the next in many applications.

Prior art document US2005/0284215 discloses a method and apparatus for the preoperative estimation of breast implant volume, in which a volume of air, water, or other substance is used to inflate one or more bladders located within the cups of a brassiere-like garment. The apparatus is operated by pumping an amount of air from the outside atmosphere, or water, or another substance from a reservoir or external water supply into the bladders, according to the patient's preference as to the size of breasts she desires. A gauge is used to measure the capacity of each bladder when inflated to its desired size. The volume of each bladder as measured by the gauge can then be used to determine the size of a breast implant to be used during breast augmentation surgery. However, the prior art does not mention how to stop inflating automatically or how to keep the object of interest to be measured immobile for measuring.

### SUMMARY OF THE INVENTION

It is among the objects of the invention to provide an apparatus which can measure the size of the object of interest and keep the object of interest still in the meantime.

To this end, the present invention provides an apparatus for monitoring an object of interest, comprising:

an elastic cushion (104);

a pump (102) arranged for inflating the cushion such that the inflated cushion extends in an inward direction so as to wrap itself around the object of interest (100);

a pressure sensor (103) arranged for detecting the pressure exerted on the object of interest (100) by the inflated cushion (104) and outputting a signal to the pump (102) to stop inflating when the pressure reaches a predefined threshold;

a measuring means (108) arranged for measuring the volume of the inflated cushion (104); and

a processor (105) arranged for deriving the size of the object of interest (100) from the volume of the inflated cushion (104).

The inflation of the elastic cushion causes the inflated cushion to expand so as to enclose the object of interest, thus keeping the object of interest still during measurements. Furthermore, the pressure exerted on the object of interest by the inflated cushion is monitored and the volume of the inflated cushion is measured, so that the size of the object of interest can be derived from the measured size of the inflated cushion and the space inside a frame that forms part of the apparatus.

In an embodiment, accordingly, the apparatus comprises a frame (101) and the cushion (104) is attached inside the frame (101).

In a further embodiment, the frame (101) further comprises a magnetic emitting means (109) arranged for emitting electromagnetic signals; a magnetic detection means (110) arranged for detecting magnetic induction signals; and an image reconstruction means (106) arranged for reconstructing images based on the detected signals and the derived size of the object of interest (100). Inputting the derived size of the object of interest into the magnetic induction tomography system enables the apparatus according to the invention to improve the quality of the reconstructed conductivity distribution image.

In another embodiment, the frame (101) comprises at least two sections that can be disassembled or opened to allow the object of interest (100) to get into or out of the frame (101). The flexible configuration of the frame makes it very convenient to house the object of interest for the purpose of monitoring.

It is another object of the invention to provide a method of measuring the size of the object of interest while keeping the object of interest still at the same time.

To this end, this invention provides a method of monitoring an object of interest, comprising steps of:

inflating an elastic cushion such that the inflated cushion extends in an inward direction so as to wrap itself around the object of interest (100);

measuring the pressure exerted on the object of interest (100) by the inflated cushion (104) and outputting a signal to stop inflating when the pressure reaches a predefined threshold;

measuring the volume of the inflated cushion (104); and

deriving the size of the object of interest (100) from the volume of the inflated cushion (104).

Modifications and variations of the apparatus and method may be carried out by those skilled in the art on the basis of the present description and figures without departing from the scope of the invention.

### DESCRIPTION OF THE DRAWINGS

The above and other objects and features of the present invention will become more apparent from the following detailed description considered in connection with the accompanying drawings, in which:

FIG. 1 is a schematic sectional diagram of an embodiment of the apparatus in accordance with the invention.

FIG. 2 schematically shows the change of the cushion in different inflation stages.

FIG. 3 is a schematic diagram of an embodiment of the frame in different operational states.

FIG. 4 is a schematic sectional diagram of another embodiment of the apparatus in accordance with the invention.

FIG. 5 is a flowchart showing the method in accordance with the invention.

The same reference numerals are used to denote similar parts throughout the figures.

### DETAILED DESCRIPTION

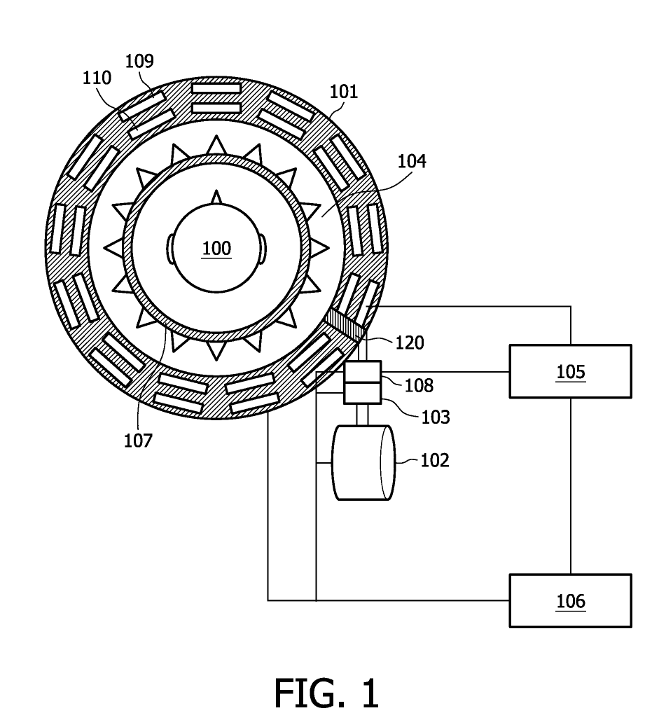

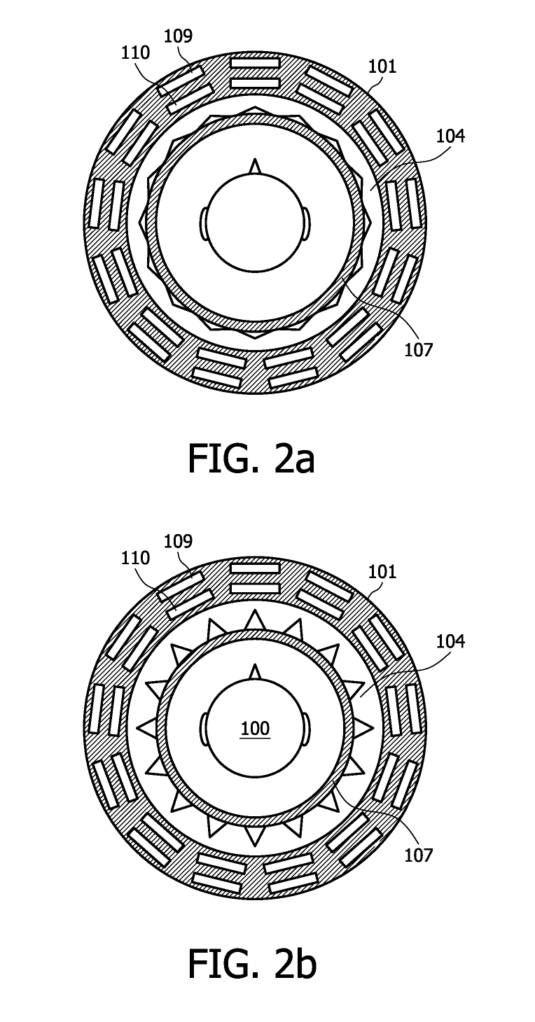

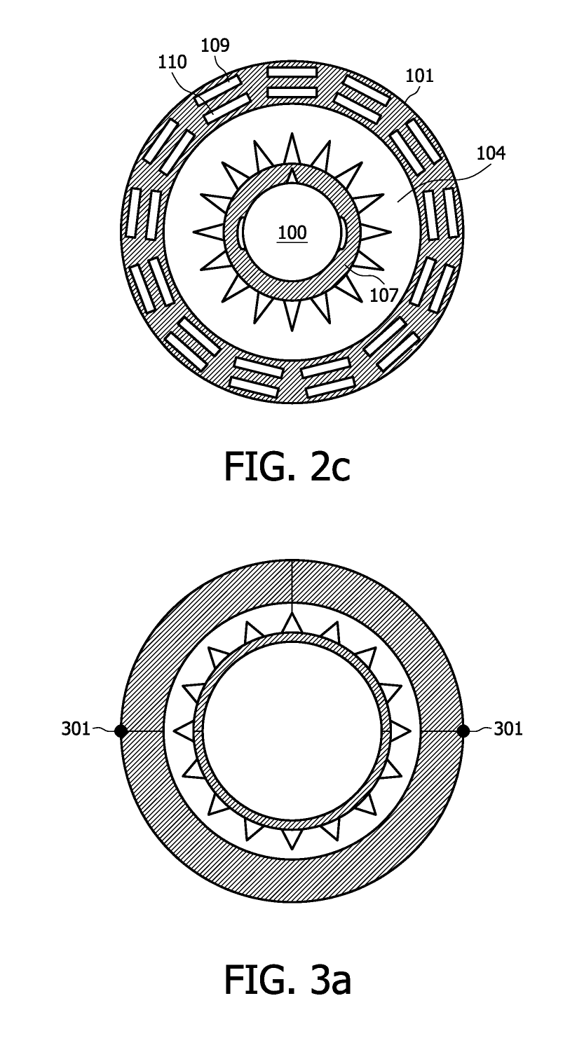

FIG. 1 is a schematic sectional diagram of an embodiment of the apparatus for monitoring an object of interest (100) in accordance with the invention. FIG. 2 schematically shows the change of the cushion in different inflation stages.

According to FIG. 1, an embodiment of the apparatus comprises:

an elastic cushion (104);

a pump (102) arranged for inflating the cushion such that the inflated cushion extends in an inward direction so as to wrap itself around the object of interest (100);

a pressure sensor (103) arranged for detecting the pressure exerted on the object of interest (100) by the inflated cushion (104) and outputting a signal to the pump (102) to stop inflating when the pressure reaches a predefined threshold;

a measuring means (108) arranged for measuring the volume of the inflated cushion (104); and

a processor (105) arranged for deriving the size of the object of interest (100) from the volume of the inflated cushion (104).

Optionally, the apparatus further comprises a frame (101), and the cushion (104) is attached inside the frame (104).

The frame 101 is most likely to be shaped as a column or a cone without top, for example a helmet shape, and the shape of the frame can be adapted to different applications. The frame 101 comprises a channel 120 having two open ends, one of the open ends is inserted into the cushion and the other is connected to the pump 102.

The cushion 104 is elastic and is accommodated inside the frame 101. The pump 102 is most suitably arranged outside the frame 101. The pump 102 is arranged to inflate the cushion 104 via the channel 120 such that the inflated cushion extends inwards to wrap itself around the object of interest 100. It should be noted that the channel 120 is not a must and that it may be replaced by a pipe to bypass the frame 101.

The shape of the cushion 104 is different with different degrees of inflation. FIGS. 2(a), (b) and (c) show the respective shapes of the cushion before inflation, partially inflated, and fully inflated.

The pressure sensor 103 is attachable to the pump and arranged to measure the pressure of the air or fluid pumped into the cushion 104, which is the same as the pressure exerted on the object of interest 100 by the inflated cushion 104. Once the pressure has reached a predefined threshold, for example a predefined pressure high enough to help keep the object of interest 100 while still keeping a patient feel comfortable, the sensor 103 outputs a signal to the pump to stop inflating.

The measuring means 108 is attachable to the pump 102. When the cushion is fully inflated, the volume of the air or fluid pumped into the cushion 104 can be measured by the measuring means 108. If the substance pumped into the cushion 104 is a liquid, the measured volume of the liquid represents the volume of the inflated cushion. If case the substance pumped into the cushion is air, the volume of the inflated cushion 104 can be derived from the measured volume of the air, the measured pressure of the air in the inflated cushion 104, and the known temperature of the air.

The processor 105 is arranged to derive the size of the object of interest 100 from the volume of the inflated cushion 104 and the volume of the space inside the frame 101. In general, the shape of the frame 101 is known and thus the volume of the space inside the frame is known or can be measured before operation. Furthermore, the gap between the inflated cushion and the object of interest can be calibrated, and the volume of the gaps can be estimated for deriving the size of the object of interest. If the apparatus does not comprise a frame 101, the predefined shape and size of the inflated cushion 104 can help to calculate the size of the object of interest 100.

Optionally, the apparatus may comprise an elastic tissue 107. The tissue is attached to the inside of the cushion and is so elastic that it can be adapted to the size of the object of interest 100 when the cushion 104 is inflated. The tissue is replaceable for individual use, providing good hygienic conditions.

In an embodiment, the frame 101 further comprises a magnetic emitting means 109 arranged for emitting electromagnetic signals; a magnetic detection means 110 arranged for detecting magnetic induction signals; and an image reconstruction means 106 arranged for reconstructing images based on the detected signals and the derived size of the object of interest 100.

The magnetic emitting means 109 and detection means 110 may comprise a plurality of respective elements, for example coils, which are accommodated in the frame 101 as shown in FIG. 1. The image reconstruction means 106 is most suitably arranged outside the frame 101 and may be integrated in a processor 105. The size of the object of interest 100 derived by the processor 105 may be used as a parameter for reconstructing the image. Furthermore, the size may be used for adjusting the magnetic emitting means 109.

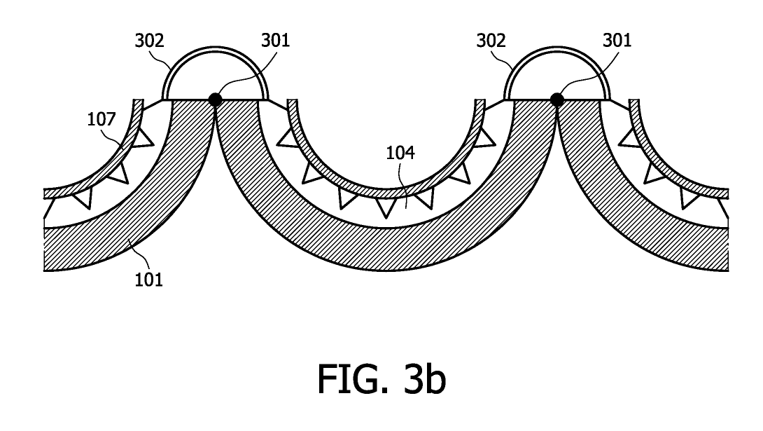

FIGS. 3(a) and (b) show the closed and open states, respectively, of an embodiment of the frame. The frame 101 comprises at least two sections that can be disassembled or opened to let the object of interest 100 into or out of the frame 101. Every two neighboring sections may be connected by hinges 301.

The apparatus further comprises a flexible pipe 302 for every two neighboring sections. The pipe 302 has two open ends which are inserted into respective neighboring sections of the cushion 104 such that the pressure in each section of the cushion 104 is uniform during inflation.

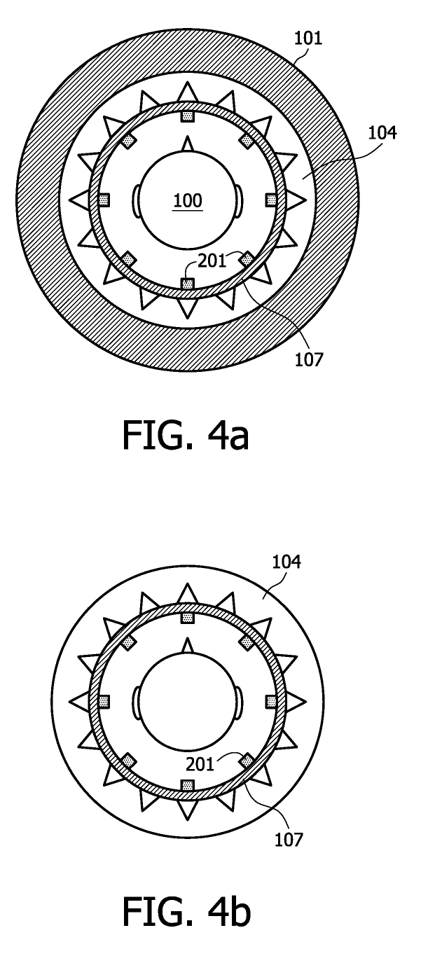

FIG. 4 is a schematic sectional diagram of another embodiment of the apparatus in accordance with the invention. FIG. 4(b) differs from FIG. 4(a) in that the frame 101 is absent. It should be noted the equivalent part outside of the frame is not shown in the diagram and that the operational scheme is similar to that of the embodiment described in FIG. 2.

In FIGS. 4(a) and (b), the apparatus comprises an ultrasound transducer 201 attached to the inside of the cushion 104. Optionally, the apparatus may comprise an additional cushion 117 which is arranged inside the cushion 104. When the cushion 104 is inflated, the cushion 117 moves inward and comes into contact with the object of interest 100, so that the size of the object of interest can be measured while the object of interest 100 is kept still.

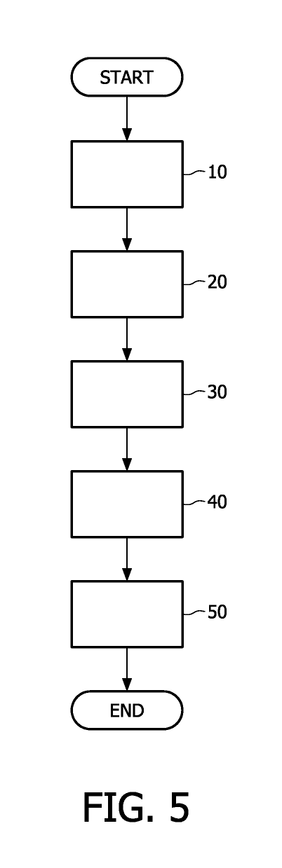

FIG. 5 is a flowchart showing the method in accordance with the invention. In step 10 of the process, a cushion (104) is inflated such that the inflated cushion extends in an inward direction so as to wrap itself around the object of interest (100). In step 20, the pressure exerted on the object of interest (100) by the inflated cushion (104) is measured, and a signal is output to stop inflating when the pressure reaches a predefined threshold. In step 30, the volume of the inflated cushion is measured. In step 40, the size of the object of interest (100) is derived from the volume of the inflated cushion (104) and the volume of the space inside the frame (101) accommodating the cushion (104).

In an embodiment, the method further comprise step 50 in which electromagnetic signals are emitted, magnetic induction signals are detected; and images are reconstructed based on the detected signals and the derived size of the object of interest.

Those skilled in the art will appreciate that the apparatus and method may be applied to different medical systems for monitoring an object of interest and acquiring image data by various acquisition techniques such as, but not limited to, Computed Tomography (CT), Magnetic Resonance Imaging (MRI), Ultrasound (US), Positron Emission Tomography (PET), Single Photon Emission Computed Tomography (SPECT), and Nuclear Medicine (NM).

It should be noted that the above-mentioned embodiments illustrate rather than limit the invention and that those skilled in the art will be able to design alternative embodiments without departing from the scope of the appended claims In the claims, any reference signs placed between parentheses shall not be construed as limiting the claim. The word “comprising” does not exclude the presence of elements or steps not listed in a claim or in the description. The word “a” or “an” preceding an element does not exclude the presence of a plurality of such elements. The invention may be implemented by means of hardware comprising several distinct elements and by means of a programmed computer. In the system claims enumerating several units, several of these units may be embodied by one and the same item of hardware or software. The usage of the words first, second and third, etcetera, does not indicate any ordering. These words are to be interpreted as names serving to identify the relevant items.

1. An apparatus for monitoring an object of interest (100) comprising:
an elastic cushion (104);
a pump (102) arranged for inflating the cushion such that the inflated cushion extends in an inward direction so as to wrap itself around the object of interest (100);
a pressure sensor (103) arranged for detecting the pressure exerted on the object of interest (100) by the inflated cushion (104) and outputting a signal to the pump (102) to stop inflating when the pressure reaches a predefined threshold;
a measuring means (108) arranged for measuring the volume of the inflated cushion (104); and
a processor (105) arranged for deriving the size of the object of interest (100) from the volume of the inflated cushion (104).

2. An apparatus as claimed in claim 1, further comprising a frame (101), with the cushion (104) being attached inside said frame (104).

3. An apparatus as claimed in claim 2, wherein the frame (101) further comprises a magnetic emitting means (109) arranged for emitting electromagnetic signals; a magnetic detection means (110) arranged for detecting magnetic induction signals; and an image reconstruction means (106) arranged for reconstructing images based on the detected signals and the derived size of the object of interest (100).

4. An apparatus as claimed in claim 3, wherein the processor is further arranged for outputting a signal based on the size of the object of interest (100) for the purpose of adjusting the magnetic emitting means (109).

5. An apparatus as claimed in claim 2, wherein the apparatus further comprises ultrasound transducers (201) for measuring ultrasound signals.

6. An apparatus as claimed in claim 5, wherein the apparatus further comprises an additional cushion (117) attached to inside of the cushion (104), and the ultrasound transducers (201) are attached to the additional cushion (117).

7. An apparatus as claimed in claim 2, wherein the frame (101) comprises at least two sections that can be disassembled or opened to allow the object of interest (100) to be moved into or out of the frame (101).

8. An apparatus as claimed in claim 7, wherein the apparatus comprises a flexible pipe (302) having two open ends for two respective neighboring sections, which open ends are inserted into said two respective neighboring sections of the cushion such that the sections of the cushion (104) are at a uniform pressure during inflation.

9. An apparatus as claimed in claim 1, wherein the apparatus comprises an elastic tissue (107) attached to the inside of the cushion, which tissue (107) is replaceable for individual use.

10. An apparatus as claimed in claim 1, wherein the substance pumped into the cushion (104) by the pump (102) for inflation is air or a liquid.

11. A method of monitoring an object of interest (100), comprising steps of:
inflating an elastic cushion such that the inflated cushion extends in an inward direction so as to wrap itself around the object of interest (100);
measuring the pressure exerted on the object of interest (100) by the inflated cushion (104) and outputting a signal to stop inflating when the pressure reaches a predefined threshold;
measuring the volume of the inflated cushion (104); and
deriving the size of the object of interest (100) from the volume of the inflated cushion (104).

12. A method as claimed in claim 11, further comprising steps of:
emitting electromagnetic signals;
detecting magnetic induction signals; and
reconstructing images based on the detected signals and the derived size of the object of interest (100).
